(19) United States
(12) Patent Application Publication
(10) Pub. No.: US 2025/0241586 A1
(43) Pub. Date: Jul. 31, 2025

(54) Bioavailability and bioequivalence through intrinsic activity and KD measurements, in vivo, in humans

(71) Applicant: Debasish Banerjee, Kolkata (IN)

(72) Inventor: Debasish Banerjee, Kolkata (IN)

(21) Appl. No.: 18/403,822

(22) Filed: Jan. 4, 2024

(51) Int. Cl. A61B 5/00

(52) U.S. Cl. CPC A61B 5/4848; A61B 5/4866; A61B 2503/40

(57) ABSTRACT

Bioequivalence and/or bioavailability data are mandatory for filing of NDA and ANDA. Presently in absence of proper procedures estimates of these data through peak plasma level concentrations and time to reach peak plasma levels are used. The method suffers from the serious drawback that peak plasma levels do not correlate with efficacy due to delay in transfer to tissue sites as well as differences in metabolic patterns in drugs. Here we have devised a novel method for determination of bioavailability and bioequivalence from measurement of threshold times and concentrations, intrinsic activity, and dissociation constants in vivo in humans. The method essentially determines Intrinsic activities and threshold doses through measurement of drug responses in humans and then uses a mathematical expression to determine KD values of drugs, in vivo, in humans. The process may be applied to animals as well. The method would go a long way in evaluation of true bioavailability and bioequivalence and would also be useful in design.

### CROSS REFERENCE TO RELATED APPLICATIONS

PCT/IB2021/057308 dated 8 Sep. 2021

### STATEMENT REGARDING FEDERALLY SPONSORED RESEARCH OR DEVELOPMENT

Not applicable

### NAMES OF PARTIES TO A JOINT RESEARCH AGREEMENT

Not applicable

### SEQUENCE LISTING

Not applicable

### STATEMENT REGARDING PRIOR DISCLOSURE BY INVENTOR OR JOINT INVENTOR

918/KOL/2010 dated Aug. 17, 2010

### BACKGROUND OF THE INVENTION

### Patent Classification Number

Methods for in vitro determination of KD values existed. U.S. Pat. No. 6,808,938 B2 shows a method for determination of KD values using biosensors, but they do not apply directly to in vivo situations (1). There are huge differences. Even in vivo cell culture values of KD (1) do not apply to in vivo in humans. Worldwide attempts were made to calculate in vivo values in live animals and humans, using radio labelled drugs. They suffer from two major drawbacks. It was found radio labelled antibodies alter receptor binding compared to non labelled compounds (2). The literature also notes that 3H labelled alprazolam does not bind to the peripheral benzodiazepine receptors (3). The second principal drawback is that labelled compounds leave traces of radioactivity in human brain kidneys etc. and human experiments are ruled out. Hence this method for in vivo determination of drug activity parameters was developed.

### BRIEF SUMMARY OF THE INVENTION

This invention relates to the measurement of bioavailability and bioequivalence of drugs in real terms in the living human body. Presently only estimates of these parameters are available through the measurement of plasma concentrations over time periods, it being assumed that plasma concentrations correlate to bioavailability, an assumption that not always holds true. The present invention uses drug concentrations at the receptor site and drug responses to obtain intrinsic activities and KD values for comparison of effectiveness of drugs in humans in vivo. Prior to this, these values were only available in isolated tissues or cell cultures and never on living animals or humans. Available literature also notes that huge differences exist between in vitro and in vivo cell culture values. (1). Hence is the need for obtaining in vivo values for KD and intrinsic activity, in humans. Here as examples of the method used, and as a demonstration of the procedure, we have obtained the values for glipizide, glimepride and alprazolam, using an equation derived for the purpose.

### BRIEF DESCRIPTION OF THE DRAWING

FIG. 1. Time Response curves for glipizide, Series 1-glucose base line, Series 2-glipizide 20 mcg/kg body weight, Series 3-glipizide 40 mcg/kg body weight, Series 4-glipizide 65 mcg/kg body weight. All values expressed as % rise or fall of blood sugar over fasting blood sugar. X axis time in mins, Y axis % rise or fall of blood sugar.

FIG. 2. Identical values for glimepride: Series 1-glucose base line, Series 2, 3 and 4, correspond to doses of glimepiride of 5, 10 and 20 mcg/kg body wt.

FIG. 3. Log dose response curve for glipizide. X-axis log dose, Y axis response.

FIG. 4. Identical values of glimepride.

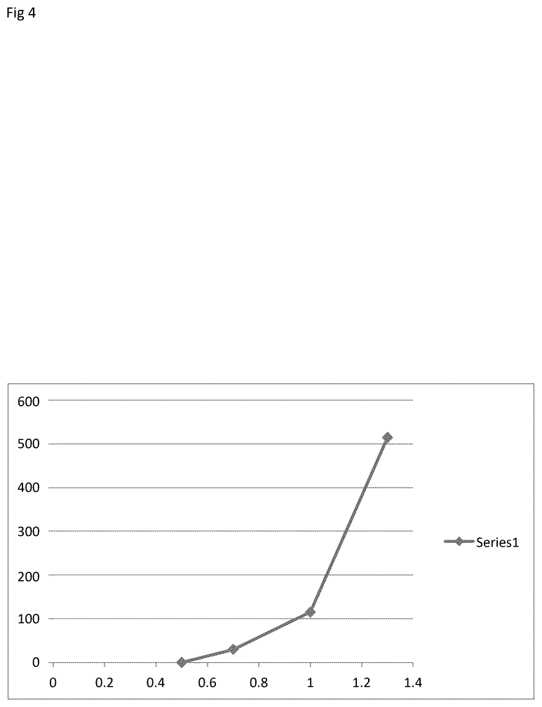

### DETAILED DESCRIPTION OF THE INVENTION

Bioavailability and bioequivalence studies are of primary importance in design of drugs and it is mandatory that such data be submitted along with every new drug application or abbreviated new drug application. Hitherto in vivo bio availability and bioequivalence values were not available and estimates of the same were obtained through plasma concentration values and time to attain peak concentration. The current method enables determination of bioavailability and bioequivalence by measuring KD and intrinsic activity at the receptor site in humans, and therefore gives a true picture in determining efficacy or equivalence of two molecules or dosage forms. It is generally accepted that onset times and peak plasma concentrations do not always reflect drug action at extra vascular receptor sites although in certain cases they are seen to correlate.

Bioavailability of a new drug or a new combination in formulation is determined by its effectiveness at the receptor site where it produces the desired effect. Although peak plasma concentration does give an estimate of the quantity of the drug reaching the receptor it has been seen that unless the drug acts on the vascular system, there are significant differences in time related blood levels and intensity and duration of pharmacological effect, particularly those arising from transfer time of drugs to the tissue and receptor sites, and from differences in metabolism. On a similar basis bioequivalence of drugs or dosage forms cannot be established by peak plasma levels and time to reach the peak plasma concentrations unless the drugs act on the vascular system. Here we have established a method for determining the bioavailability and bioequivalence of drugs and formulations by the study of intrinsic activities, threshold doses and times, and KD values in humans in vivo in living systems. The method is superior since it works through direct real time measurement of drug effect and not by indirect means where correlation between peak plasma concentration and effect is not certain.

Drug action is measured by the following parameters all measured in vivo in living animals/humans.
A) Onset time and threshold dose/concentration (minimum dose/concentration to initiate the effect,) these indicate the potency of the drug.
B) Intrinsic Activity or the maximum possible effect,
C) Dissociation constant. Its reciprocal measures the affinity of the drug for the receptor.
   The above named, parameters have been measured as stated below:

For glipizide, glimepride and alprazolam:

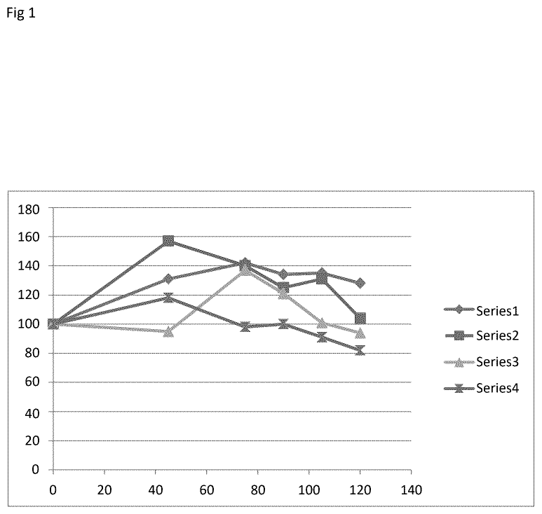

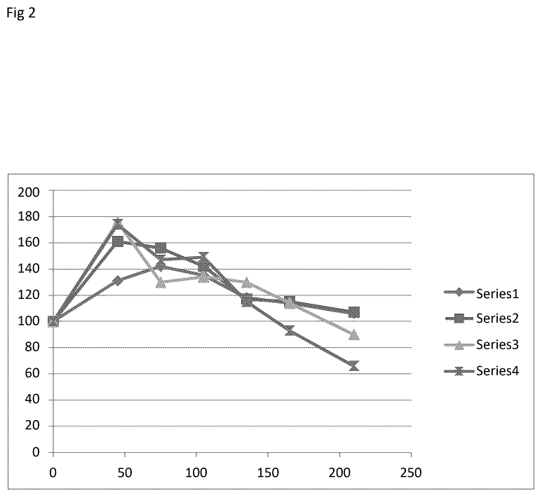

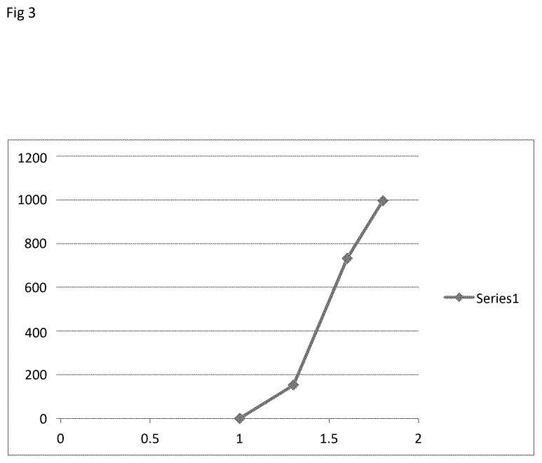

Volunteers chosen were of sound health average weight same age and gender, having normal creatinine clearance and having no hypersensitivity towards glipizide or glimepride.
1 Their fasting blood sugars were noted and were given glucose 85 gms and their blood sugar levels monitored at regular time intervals for about 3 hrs. This gave their glucose base lines.
2 They were then treated with glipizide in the under mentioned doses and blood sugar levels monitored at earlier mentioned intervals for 3 hrs. The doses were 20, 40 and 65 mcg/kg body wt.
3 The differences between the AUC's of the glucose base line and that of glipizide at various doses gave the effect of glipizide on the reduction of blood sugar levels of those individuals at the doses administered.
4 A time of 7 days was allowed between 2 subsequent doses in order to wash out the effect of the previous dose.
5 The results are shown in FIG. 1.
6 After a lapse of 7 days the same individuals were administered the following doses of glimepride and the experiment repeated. The doses were 5, 10 and 20 mcg/kg of body wt.
7 The results are shown in FIG. 2.
9 From the responses (as measured by the reduction in AUC's of different doses of glipizide and glimepride) the log dose response curves of the two drugs are constructed. (FIGS. 3 & 4)
10 The points of inflection of the log dose response curves gave the threshold doses of glipizide and glimepride
11 The free drug concentrations were calculated at threshold and higher doses and are correlated to the reciprocal of fractional responses vide the equation (1/fractional response)=1+ (KD/A) where A is the unbound drug concentration at equilibrium. Fractional response is measured by (response at that dose/maximum response or intrinsic activity).
12 Table I gives these values for glipizide
13 Table II gives the values for glimepride.
14 The slopes were used to calculate the KD values for glipizide and glimepride. The KD value obtained for glipizide was 8.98 nm and those for glimepride was 2.0 nm. The literature values (in vitro and cell culture) for glipizide were in the region of 10 nm (2). The literature also notes that there could be huge differences in in vivo and in vitro values and in such cases in vivo values are to be taken, if available (1). Existing literature gives no value for KD for glimepride. A point in this regard is that many drugs act on multiple receptor systems and in such cases the response is to be chosen such that only one receptor system is involved. For instance glipizide and glimepride act on the K+ channels of the beta cells of the pancreas.
15 The only assumption used in this calculation is that A, the unbound drug concentration is equal to the concentration of free drug at receptor site at equilibrium. The assumption is valid since the amount of bound drug is negligible compared to the total amount of drug at receptor and may be neglected. (The amount of receptor bound drugs depend on the number of free receptors, which are generally far less than the total number of drug molecules in solution.) Therefore free drug concentration almost equals the unbound drug.
16 The KD values for alprazolam were calculated as for glipizide and glimepride. Alprazolam was administered to human male volunteers with sound health, of average weight and having normal creatinine clearance in the doses of 4 mcg/kg, 6 mcg/kg and 8 mcg/kg, and onset and sleep times noted.
   The amount of free drug crossing the blood brain barrier was obtained. Since the response used in the study was sleep time the amount of drug crossing the blood brain barrier was used in the calculation instead of free drug concentration in plasma.
   The KD values were calculated from the slope of the reciprocals plot as of glipizide and glimepride. It came to 50.8 nm. (Table III). The literature has no value for KD for alprazolam. It does give a value for labelled 3H alprazolam but also states that the labelled drug is not recognised by peripheral benzodiazepine receptors. The value does not apply here. (3)
      The intrinsic activities were the maximum responses of the drugs. They were recorded along with threshold values as under:

### Intrinsic Activity:

$$\begin{matrix}Glipizide = 996 * 0.05 = 49.8 \% ( reduction ) * hr \\ Glimepride = 514 * 0.05 = 25.7 \% ( reduction ) * hr \\ Alprazolam = 241 min sleep time\end{matrix}$$

### Threshold Dose:

Glipizide 20 mcg/kg body weight
Glimepride 5 mcg/kg body weight
Alprazolam 4 mcg/kg bodyweight

### BEST MODE OF CARRYING OUT THE INVENTION

As detailed in the disclosure of the invention the methods for glipizide glimepride and alprazolam have been given. One has to realize that the methods will vary with different types of responses, different receptor systems the drugs that act on them. However this invention provides a method for comparison of different drugs and formulations acting on the same receptor system, a method that directly involves the receptor system through which the drugs act, and is far more relevant and depends on real time dissociation of the drug molecule from its pharmacological receptor system, compared to indirect methods of comparing Cmax and AUC values. Ideally an effect where the drugs acting through a single receptor system gives the desired pharmacological response, are best suited for this study, (like alprazolam in the brain or glipizide/glimepride in the pancreas).

### INDUSTRIAL APPLICABILITY

1 This process fulfils the long felt need of deriving in vivo pharmacokinetic and pharmacological parameters that would be used in comparing drugs and formulations and gives results directly applicable to in vivo life processes, such as cell signaling pathways and would be useful in predicting effects of new drug on the basis of comparisons with molecules currently in use.

2 It is non compartmental and model independent.

This process compares in vivo pharmacological effects of molecules acting through the same receptor systems, (to arrive at conclusions in respect of their equivalence or utility in life processes,) in terms of equivalence of the two drugs in respect of their biological activities, instead of arriving at conclusions based on indirect parameters such as plasma concentrations which may or may not correlate to biological activities.

### Mathematical Section:

Derivation of the equation in paragraph [0007], part 11, line 2 of page 3.

Since activity is proportional to the fraction of receptor occupied:

So

$$Activity = X * fraction of receptor occupied .$$

Where X=intrinsic (maximum) activity.

Therefore

$$( Activity at dose / maximum activity ) = ( fraction of receptors occupied ) = ( RA / r ) \qquad ( 1 )$$

Where RA=number of receptors occupied, r=total number of receptors, and R=number of free receptors, at equilibrium.

Now a drug receptor interaction is represented by the equation,

$$R * A = RA$$

Where R=number of free receptors and A=number of free drug molecules.

Let K1=rate of forward reaction, and

K2=rate of backward reaction.

At equilibrium the rates of forward and backward reactions are the same.

Therefore,

$$K 1 * R * A = K 2 * RA$$

Or,

$$( K 1 / K 2 ) = ( RA / R * A )$$

Or,

$$Ka = ( K 1 / K 2 ) = Association constant of the drug .$$

Therefore,

$$Ka = ( RA / R * A ) = [ RA / ( r - RA ) * A ] ,$$

Or

$$( 1 / Ka ) = [ ( r - RA ) * A / RA ]$$

Or

$$( 1 / Ka * A ) = ( r - RA ) / RA = ( r / RA ) - 1$$

Or

$$( r / RA ) = 1 + ( 1 / Ka * A )$$

Or

$$[ 1 / ( RA / r ] = 1 + ( 1 / Ka * A ) = 1 + ( Kd / A )$$

Where, Kd=dissociation constant.

From (1) . . .

$$( RA / r ) = fractional activity$$

Therefore

$$( 1 / Fractional Activity = 1 + ( Kd / A ) \qquad Q . E . D$$

### REFERENCES

1 Wang, M. Et. al., British Journal of Pharmacology, (2017), V-174. P-70-81
2 Edelman M. R. et. al., Journal of labelled compounds and radio pharmaceuticals, [2019], V-62(11), p-751-757.
3 K. Wamsley et. al., Am. College of Neuropsychopharmacology, (1993) Published by Elsevier Science Publishing Co, Avenue of the Americas, New York, NY 10010

TABLE I
Glipizide

| | A | | Response | |
|---|---|---|---|---|
| Dose | Free drug | | mg*hr/dL | 1/Fractional |
| Mcg/kg | Mcg/ml | 1/A | AUC reduction | response |
| 20 | 4.6/10\hat{ }4 | 2173.9 | 153 | 6.5 |
| 40 | 9.2/10\hat{ }4 | 1086.9 | 732 | 1.36 |
| 65 | 14.8/10\hat{ }4 | 675.7 | 996 | 1.0 |

Y(1/Fractional Response) = −2.25 + 3.88/10\hat{ }(3)*A
r = 0.98
Slope = 3.88/10\hat{ }3
Mol Wt = 445.5
KD = 8.98 nm

TABLE II
Glimepride

| | A | | Response | |
|---|---|---|---|---|
| Dose | Free drug | | mg*hr/dL | 1/Fractional |
| Mcg/kg | Mcg/ml | 1/A | AUC reduction | response |
| 5 | 0.45/10\hat{ }4 | 22222.2 | 30 | 17.24 |
| 10 | 0.90/10\hat{ }4 | 11111.1 | 115 | 4.48 |
| 20 | 1.80/10\hat{ }4 | 5555.4 | 514 | 1.0 |

Y (1/Fractional response) = −5.38 + 9.99/10\hat{ }4*A
r = 0.99
Slope = 9.99/10\hat{ }4
Mol Wt = 490.67
KD = 2.018 nm

TABLE III
Alprazolam

| | A | | Response | |
|---|---|---|---|---|
| Dose | Free drug** | | sleep time | 1/Fractional |
| Mcg/kg | Mcg/ml | 1/A | (min) | response |
| 4 | 0.0044 | 225.8 | 63 | 3.8 |
| 6 | 0.0066 | 151.4 | 100 | 2.4 |
| 8 | 0.0088 | 49.8 | 241 | 1.0 |

Y (1/Fractional response) = 0.1519 + 15.7/10\hat{ }(3)*A
r = 0.99
64 Slope = 15.7/10\hat{ }(3)
Mol Wt = 309
KD = 50.8 nm
**Free drug quantity crossing the blood brain barrier.

I/We claim:

1. A process for measuring parameters of drug activity, intrinsic activity, KD values, threshold times and concentrations, comprising of, direct measurements of drug effects on live humans and animals in vivo, and derivation of KD values from a mathematical expression, using the reciprocals of concentration and fractional responses plots (paragraph [0007]0.11 and mathematical section), for use in estimation and comparison of bio-availabilities and bioequivalences of drugs and formulations.

2. The process as claimed in [claim 1], for comparing the effect of drugs and formulations through direct measurements of parameters of drug activity, in vivo on humans, for use in comparisons between multiple drugs and formulations acting through the same receptor system.

3. The process as claimed in [claim 1], which is a non-compartmental, model independent method, for use in obtaining real time in vivo parameters of drugs and formulations, compared to model dependent estimates of activity based on AUC's and Cmax values.

4. The process as claimed in [claim 1], where the improvement comprises the fact that the values can be directly correlated to clinical effects in vivo. (Formed by division of claim 1).
